(19) United States
(12) Patent Application Publication
(10) Pub. No.: US 2025/0242073 A1
(43) Pub. Date: Jul. 31, 2025

(54) APPARATUS FOR TRANSMISSION OF MULTIPLE DISTINCT ODORS

(71) Applicant: Science Lab 3 Inc., Dana Point, CA (US)

(72) Inventors: Hussain Rangwala, Villa Park, CA (US); Alan Bernstein, Dana Point, CA (US); Christian N. Garnett, Mount Kisco, NY (US); Young A. Tang, Downey, CA (US); Alex D. Kirkpatrick, Saratoga, CA (US); Dawid Bogdal, Gilroy, CA (US)

(73) Assignee: Science Lab 3 Inc., Dana Point, CA (US)

(21) Appl. No.: 18/986,072

(22) Filed: Dec. 18, 2024

Related U.S. Application Data
(63) Continuation of application No. 18/423,755, filed on Jan. 26, 2024.

(51) Int. Cl. A61L 9/03; A61M 21/00

(52) U.S. Cl. CPC A61L 9/035; A61L 9/032; A61M 21/00; A61L 2209/11; A61L 2209/131; A61L 2209/133; A61L 2209/135; A61M 2021/0016

(57) ABSTRACT

An apparatus comprises a belt made of a continuous band having a plurality of scent reservoirs attached thereto. The apparatus includes a belt motor configured and positioned to drive and stop the belt. A heater block is positioned to heat one of the scent reservoirs having a first scent, after the motor positions the belt to bring one of the plurality of scent reservoirs into proximity to the heater block. A fan is positioned to blow air past the heated one of the plurality of scent reservoirs and out of the apparatus, presenting the scent to a user. The belt may then be moved to bring another scent reservoir into proximity to the heater block, this latter scent reservoir having a different scent, and the process may repeat. A series of scents may be presented to a user in this manner.

This application is a continuation of prior U.S. patent application Ser. No. 18/423,755 (filed Jan. 26, 2024), which claims the benefit of U.S. Provisional Application No. 63/481,768 (filed Jan. 26, 2023), both of which are incorporated herein by reference in their entireties.

### BACKGROUND OF THE INVENTION

### Field of the Invention

The invention described herein relates to provision of scents to a patient for therapeutic or other purposes.

### Background Art

Environmental enrichment has been shown to have a positive impact on cognitive function such as in ameliorating cognitive decline in various animal models. Enriching an environment can be accomplished in a variety of ways including, but not limited to, the introduction of various objects, sounds, smells, colors, animals, etc. Research papers reaching this conclusion have been published and the cognitive benefits have been shown to reduce or overcome animal models of human neurological disorders such as Alzheimer's disease, memory loss, vascular dementia, neuronal death in aging, traumatic brain injury, head injury, Parkinson's disease, seizures, stroke symptoms, multiple sclerosis, anxiety, autism, ADHD, Huntington's disease, Down Syndrome, stress, depression, cerebral palsy, chemo-brain disorders, schizophrenia, prenatal alcohol syndrome, lead exposure, addiction, and cancer, to name a few. Noticeable behavioral changes have been observed in children with autism after as little as six months of environmental enrichment exercises.

Of the various stimulants used in environmental enrichments, it has been found that cognition is strongly associated with olfaction. Olfaction is the only sense that has a large, direct pathway to the cognitive and emotional areas of the brain. Loss of olfaction sometimes precedes memory loss from aging and from forms of dementia. Olfactory loss triggers a significant loss of neurons in the brain. After the age of 50, it has been found that decline in olfactory ability accurately predicts all-cause mortality within the next five years.

Biologically, olfactory stimulation activates the entorhinal cortex. The entorhinal cortex diminishes with age and with other factors such as Alzheimer's and other forms of dementia. As the entorhinal cortex diminishes, it releases the dentate gyrus and CA3 (hippocampal subfields) from inhibition, thereby interfering with memory. Thus, a restoration of olfaction may increase neurogenesis and/or neural complexity in the entorhinal cortex, thereby normalizing dentate/CA3 activity and restoring memory.

Interventions based on environmental enrichment can be expensive or difficult to maintain, as in the case of exercise. Currently, there is a lack of environmental enrichment methods and apparatus that effectively improve cognitive function and memory while being low cost, and easy to use or maintain. More specifically, there is a lack of environmental enrichment equipment that exploits the strong association that olfaction has with the cognitive and emotional areas of the brain.

### BRIEF DESCRIPTION OF THE DRAWINGS/FIGURES

FIGS. 1A and 1B illustrate an embodiment as viewed from the front.

FIGS. 2A and 2B illustrate an embodiment as viewed from the rear.

FIG. 3A illustrates a top view of a scent-carrying belt in accordance with some embodiments.

FIG. 3B illustrates a side view of the scent-carrying belt of FIG. 3A.

FIG. 3C illustrates a top view of a scent-carrying belt in accordance with some embodiments.

FIG. 3D illustrates a side view of the scent-carrying belt of FIG. 3C.

FIG. 4 is an exploded view of an embodiment.

FIG. 5 illustrates a process of presenting scents to a user, according to an embodiment.

Further embodiments, features, and advantages of the present invention, as well as the operation of the various embodiments of the present invention, are described below with reference to the accompanying drawings.

### DETAILED DESCRIPTION OF THE INVENTION

A preferred embodiment of the present invention is now described with reference to the figures, where like reference numbers indicate identical or functionally similar elements. While specific configurations and arrangements are discussed, this is done for illustrative purposes only. A person skilled in the relevant art will recognize that other configurations and arrangements can be used without departing from the spirit and scope of the invention. It will be apparent to a person skilled in the relevant art that this invention can also be employed in a variety of other systems and applications.

The process of olfactory therapy can require sequential detection by a patient of a number of novel and distinct odors. This has historically been either a manual process (e.g. sniffing from sample vials) or a somewhat invasive process (e.g., using masks or nasal tubes). The challenge lies in delivering distinct odors to a patient from a distance, without oversaturating the environment with odorant. Oversaturation would make it difficult to clear one distinct odor before starting the next.

To solve this challenge, the description herein discloses embodiments of a device that use odor-infused wax, which has a large differential in odor transmission rate between its solidified and melted states. Multiple pieces of wax, each incorporating a different scent source material resulting in a different respective odor, are placed into a device that cycles through each wax piece individually, selectively heating it to above its melting point to release its odor, i.e., vapor from its scent source material, exposing it to an air current to transmit the odor to the user or patient at a distance, and then cooling the wax to below its melting point to stop the release of odor, transitioning to the next wax piece, and repeating. The disclosed embodiments can provide therapy that may be useful for treating memory degradation and other neurological conditions. Note that in this document, the terms “vapor” and “vaporization” are used broadly to refer to any heat-facilitated release of molecules that constitute a scent, where the release may not necessarily require a discrete phase change to a gaseous state.

Embodiments of the apparatus and processes include a number of features, listed here and detailed below, but are not limited to these features:

Selective exposure, to a current of air, of a single scent among a multitude of scents; and repeating for each of a sequence of scents.

Selective exposure to the environment of a single scent among a multitude of scents; and repeating for each of a sequence of scents.

Selective heating and melting of a wax piece, with its incorporated scent source material, from among a multitude of wax pieces; and repeating for each of a sequence of wax pieces.

A series of scent reservoirs containing odor-infused wax pieces, arranged on a closed-loop belt with the opening of each reservoir facing towards the center of the loop.

A membrane to seal off the opening of each scent reservoir, which allows odor to pass through but prevents wax from passing through.

A pair of drums, around which the belt of scent reservoirs is wrapped. An upper drum engages with features on the belt and rotates to function as a drive wheel to move the belt around the two drums. A lower drum is hollow, does not rotate, and has an opening on its bottom face to expose the opening of a single reservoir to the center of the lower drum.

A fan, positioned at the rear of the lower drum, which blows air through the lower drum, over the opening of a single scent reservoir, and out the front of the device.

A heating element, which raises and lowers to surround the single scent reservoir that is exposed to the center of the lower drum, in order to heat the contents of the scent reservoir.

An enclosure, which isolates all the scent reservoirs from the user environment except for the reservoir that is presently aligned with the opening on the bottom of the lower drum.

A door, which opens the enclosure to allow for installation and replacement of the belt and for maintenance of other internal components.

One or more control modules, programmable to create specific schedules and sequences of scent delivery.

The apparatus described herein allows for a multi-pronged approach to scent delivery and scent isolation, enabling environmental isolation, airflow isolation, and thermal isolation. A device could selectively expose one scent at a time to the user environment using one or more of or a combination of an isolation chamber, directed air stream, and directed heat source. By combining scent isolation, environmental isolation, airflow isolation, and thermal isolation, the apparatus described here provides the necessary combination of fast detection, fast clearing, clear differentiation, and minimal accumulation of scents.

Additional detail is now provided with respect to the figures presented herewith.

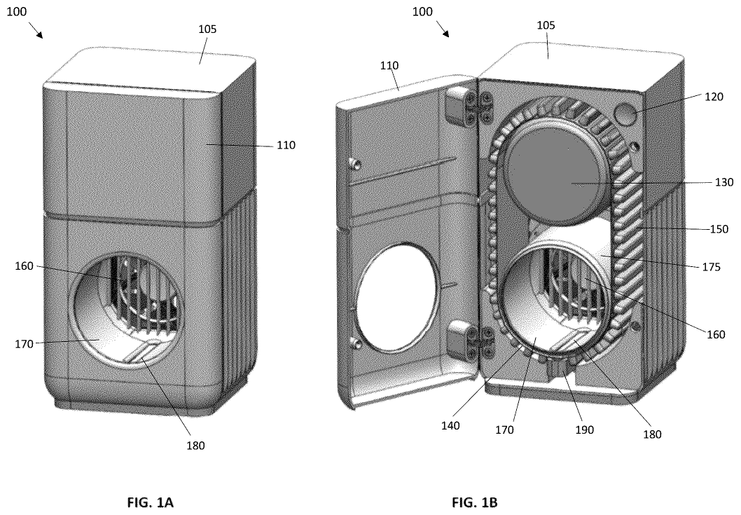

FIG. 1A shows a device 100 for providing a series of scents, according to an embodiment. The device may include a rear enclosure 105 and an access door 110 that may be opened as necessary for maintenance purposes. The device 100 may include an airflow chamber 170 which is a volume of space formed by the interior of a lower drum, as will be described in greater detail below. At the bottom of airflow chamber 170 is an opening 180 that exposes the contents of a scent reservoir. The device 100 may include a series of scent reservoirs, at least some of which may provide a respective distinct scent. For a scent reservoir positioned beneath the opening 180, vapor of the scent may be released through the opening 180 and into the airflow chamber 170. A fan 160 at the rear of device 100 may then blow the vapor out of the airflow chamber 170. As used, the device 100 may used to direct the vapor to a user. The airflow chamber 170 also serves as an isolation chamber, in that a single scent is introduced into the chamber at any given time. The scent is cleared from the chamber by operation of the fan 160 before any further scent is introduced.

Note that in some applications, the user may be a patient for which the series of scents may have therapeutic value.

Some of the internal components of device 100 are shown in FIG. 1B according to an embodiment. These components may include a start button 120. These components may further comprise a closed-loop belt 140. The belt 140 may be a band to which the series of scent reservoirs 150 is attached. Each reservoir may be spaced apart from each other on belt 140, and each reservoir may contain a different scent. In some embodiments, no two adjacent reservoirs contain the same or similar scent. The band may be made of a durable yet flexible material, discussed in greater detail below. In the illustrated embodiment, the belt 140 may be moved by a rotatable upper drum that effectively serves as a drive wheel 130. The belt 140 may therefore be moveable in a looping path around a portion of lower drum 175. The lower drum 175 has an opening 180 at the bottom in the illustrated embodiment. Belt 140 may also be moved by lower drum 175. Although not shown, in one embodiment, device 100 may comprise a single rotatable drum such as drive wheel 130. In this embodiment, belt 140 is configured to partially or fully wrap around the single rotatable drum. Other than the two-drum aspect of device 100, all other features and functions as described herein can be implemented in the single drum device.

In operation, drive wheel 130 is configured to move the belt 140 in a manner that positions one of the scent reservoirs 150 near the opening 180 and in proximity to a heater block 190. In some embodiments, drive wheel 130 is configured to rotate the belt 140 such that one of the scent reservoirs 150 is located below the opening 180. Once one of the scent reservoirs 150 is positioned below the opening 180, the motion of belt 140 may be paused for a desired duration In this way, heater block 190 can heat up scent reservoir 150 and thereby causing a scent embedded in reservoir 150 to be released for a determined amount of time. In some embodiments, the heater block 190 may be composed of metal, metal composite, ceramics, and/or other materials in combination. In an embodiment, the heater block 190 may be raised or moved into a position closer to the now stationary scent reservoir 150, and heat may be applied by the heater block 190. In an embodiment, the heater block 190 may be recessed so that it may surround the scent reservoir 150 to provide efficient heat transfer.

In some embodiments, a heating rod (not shown) can be implemented in place of heater block 190. The heating rod can be positioned near or just above the opening 180. Similar to hearing block 190, heating rod may be composed of metal, composite, ceramics, or a combination thereof.

In an embodiment, the scent reservoir 150 holds a piece of wax that incorporates a scent source material, such as a scented oil or other scent-bearing material. Heating the wax allows the scent within the scent-bearing material to be released. In some embodiments, the scent-bearing material may also be heated when the wax is heated. When heated, the scent-bearing material may undergo a heat-induced change in one or more of its material characteristics, thereby releasing the scent. In some embodiments, the scent-bearing material is the wax itself. When heated, the wax can partially or fully melt and cause the scent to be released. Scent or vapor from the wax and/or the scent-bearing material then enters the interior of the lower drum 175 when heat is applied for a predetermined heating interval. The interior of the lower drum 175 is shown in FIGS. 1A and 1B as airflow chamber 170. The fan 160 may be activated to push the vapor through the airflow chamber 170 and out of the device 100. The device 100 may be positioned such that the vapor is directed towards a user.

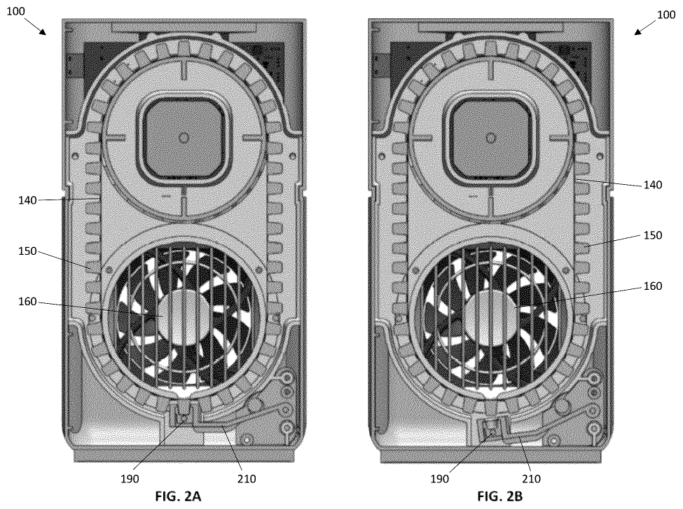

FIGS. 2A and 2B show the rear of device 100, according to an embodiment. In both figures, belt 140 is shown having a series of scent reservoirs 150 attached thereto. In an alternative embodiment, the scent reservoirs may be integrated or embedded into the belt. The fan 160 is shown in a position to draw air in from the back of device 100. FIG. 2A shows a heater block 190 positioned to heat one of the scent reservoirs 150. In some embodiments, the heater block 190 can be positioned such that it encompasses a portion of reservoir 150. The heater block 190 can be positioned by a connected lever arm 210. Alternatively, the heater block 190 can be positioned by other mechanical means such as an axial actuator (not shown) positioned below reservoir 150.

After a heating interval has been completed, the lever arm 210 and heater block 190 may move away from the scent reservoir as shown in FIG. 2B. In this way the wax piece in the scent reservoir may be allowed to cool. In an embodiment, as the wax piece cools, the belt 140 may rotate so that the cooling scent reservoir moves away and a different scent reservoir may be put into place for heating.

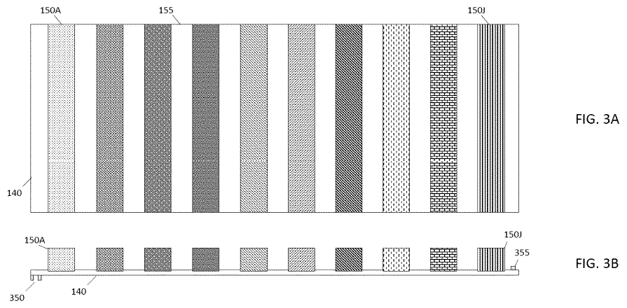

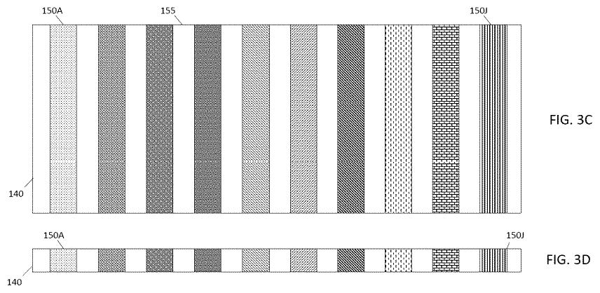

FIG. 3A illustrates a section of an embodiment of belt 140 having a plurality of scent reservoirs 150A through 150J. While this embodiment shows ten scent reservoirs on the belt, other embodiments may have more or fewer. Each reservoir can contain a different scent. Belt 140 may alternatively contain one or more repeating scents. In some embodiments, no two adjacent reservoirs contain the same or similar scent. Scent reservoir 150 may be disposed on a surface of belt 140 as illustrated in FIG. 3B. Additionally, belt 140 includes scent free zones 155 disposed between adjacent scent reservoirs 150. In some embodiments, scent free zones 155 can contain or be infused with scent neutralizers. Each scent free zone 155 can contain different scent neutralizers configured to neutralize a specific scent. For example, one of the free zones 155 can have neutralizer specifically formulated to neutralize a base scent of a fragrance pyramid. Another free zone 155 can have a different neutralizer specifically formulated to neutralize a top scent. Scent neutralization is discussed further below.

Belt 140 may be made of two or more belt sections that each include attachment features 350 and 355 at each end of the belt section. In this way belt sections can be fastened to each other to form a continuous band. Attachment features 350 and 355 may be disposed on one of the surfaces of each belt section. Alternatively, attachment features 350 and 355 may be disposed at the side edge at the very end of each belt section. Attachment features 350 and 355 may be a male-female fastening feature, a hook and loop fastening feature, or other fastening means such as magnets and adhesives.

In an embodiment, the belt 140 may be made of four belt sections connected end-to-end using the attachment features, forming a closed loop. The belt sections may be made of a plastic or polymeric material, such as polypropylene, high density polyethylene, or high impact polystyrene, as examples without limitation.

In another embodiment, the belt may not be a loop, but may instead be linear. In this case, an alternative mechanism (not shown) may be used to move the belt back and forth as necessary to position its scent reservoirs in proximity to a heating element.

FIGS. 3C and 3D illustrate an embodiment of belt 140 where the plurality of scent reservoirs are embedded within belt 140. Rather than being raised above the surface of belt 140, scent reservoirs 150 of this embodiment are integrated within belt 140.

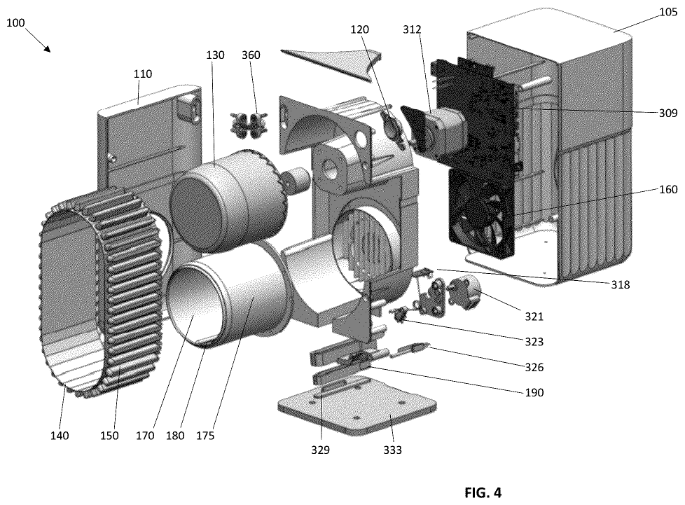

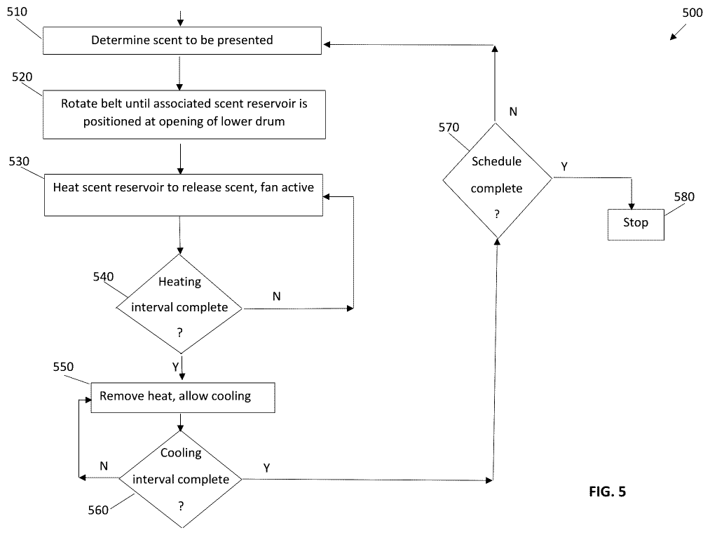

FIG. 4 shows device 100 in an exploded view. Belt 140, with its scent reservoirs 150, are mounted on a motorized drive wheel 130 and on the stationary lower drum 175. The motion of the drive wheel 130 and the belt 140 may be determined by a drive wheel motor 312. The drive wheel motor 312 may be controlled by a control module. The control module may be implemented as logic embodied on a main printed circuit board (PCB) 309. In various embodiments, this logic may be implemented in hardware, firmware, software, or a combination thereof. This logic may control when the drive wheel turns, how far it turns, when it pauses, and the length of the pause. This logic may also control the duration of each heating interval and/or the intensity (i.e., temperature) of the heating process. In this way, the logic of PCB 309 may specify the particular scent reservoir 150 that is heated, the duration and/or temperature of the heating, the duration of the cooling, and the sequence of scent reservoirs 150 (and the corresponding sequence of scents) that are presented to a user. The logic may also specify the higher level scheduling of operation of device 100, i.e., the timing and duration of a sequence of scent presentations. For example, the logic may specify that scent presentations take place every night during hours when the user is normally in bed. In another embodiment, the logic may be responsive to biometric sensors that measure physiological parameters of the user, so that scent presentation only takes place when these parameters indicate one or more particular stages of sleep.

PCB 309 can also include logic to control the position of the heater block arm 210 (cam) or the axial actuator (not shown) based on a scent presentation schedule, heating internal and/or intensity. For example, during a scent presentation mode, PCB 309 may include logic to move the heater block 190 into place and turn on heater block 190. During a break period between scent presentations, PCB 309 may include logic to move the heater block 190 away from scent reservoir 150 and/or turn off the heater block 190.

To facilitate control of the belt 140, a belt position sensor 318 may be used to detect the position of the belt 140 and to provide this information to the logic of PCB 309. The position of belt 140 may be sensed by an optical and/or mechanical process. For example, the belt position sensor 318 may optically detect physical or identifying markers on the belt 140 and/or on the scent reservoirs 150 to determine the position of the belt. Such identifying markers can be a bar code, QR code, NFC (near field communication) chip, NFC tag, RFID (radio frequency identifier) tag, or low energy Bluetooth beacon associated one or more scent reservoirs 150 or each belt 140, for example and without limitation. The identifying marker can identify one or more of: the position of a scent reservoir with respect to the isolation chamber 170; the scent type (e.g., peach, vanilla, cinnamon, mint) of each scent reservoir on belt 140; the scent identifying number, which can also provide the scent type and other characteristics (e.g., recommended neutralizer); and the order of the scent as positioned on belt 140 . . . . Alternatively the belt position may be determined by tracking the degree and direction in which the belt has moved with respect to a known reference point. In an embodiment, the logic of PCB 309 may be programmable by a manufacturer, a medical care provider, and/or a user. The interface for such a programming process may be wired or wireless and use any communications protocol known to persons of ordinary skill in the art. In an embodiment, the programming may be performed through an application running on a user's computing device, such as a personal computer, laptop, tablet, smartphone, or other user device.

As discussed above, a wax piece may be heated by a heater block 190. The heater block 190 may be covered on its underside by an insulated heater block cover 329. Further insulation may be provided by heater block arm 210. The heater block 190 may be heated by a heating element, shown here with a thermistor and thermostat. These three components are shown collectively as assembly 326. The heater block 190 may be moved into and out of position by a heater block motor 321. Positioning of the heater block 321 may be further controlled by a limit switch 323.

Operation of device 100 includes the following actions, according to an embodiment. The logic of PCB 309 may first be programmed by a user, caregiver, retailer, or manufacturer to specify a sequence or rotation of particular scents that device 100 is to present. The programming may also specify the duration of presentation of each scent and/or a time interval between scents.

Different scents in the programmed sequence correspond to respective scent reservoirs 150 on belt 140. The sequence of scents therefore corresponds to a series of scent reservoirs 150 that are to be positioned below opening 180. This in turn corresponds to a series of positions of belt 140, where each position corresponds to the appropriate scent reservoir 150 positioned below the opening 180. A particular belt position may therefore be identified by the logic running on PCB 308. Under control of this logic, drive wheel motor 312 may rotate the drive wheel 130 to position belt 140 so that the desired scent reservoir 150 is beneath the opening 180 of the lower drum 175. Drive wheel 130 and belt 140 may then halt. The required positioning of the belt 140 may be facilitated by the belt position sensor 318.

Once stationary, the scent reservoir 150 is in proximity to the heater block 190, and may then be heated by heater block 190, by raising the lever arm 210 so that the heater block 190 is adjacent or sufficiently close to the scent reservoir 150, and activating heating element assembly 326. This melts, at least partially, the wax piece of scent reservoir 150 and vaporizes some of the scent source material therein, releasing the vapor into airflow chamber 170.

In an embodiment, the scent reservoir 150 may include a membrane or other barrier (not shown) that allows the vapor to pass out of the scent reservoir 150 while maintaining the wax in place. The fan 160 may push air over the opening 180 to move the scent out of device 100 and toward a user.

Once the scent has been delivered for a time interval specified by the programming, the lever arm 210 may lower, moving the heater block 190 away from the scent reservoir 150, which may then be allowed to cool. In an embodiment, the lever arm 210 may be spring loaded to raise the heater block 190, and a rotating cam may be used to lower the lever arm 120, moving it from the scent reservoir 150. A next scent (and associated scent reservoir 150) may be specified by the programming, and the process may be repeated for this latter scent reservoir, and for any further scents and scent reservoirs. The motion of the lever arm 210 and heater block 190 towards and away from the stationary scent reservoir 150 may be actuated by a mechanism connected to heater block motor 321 and comprising one or more cams, as would be understood by a person of ordinary skill in the art.

In the illustrated embodiment, the above components are housed in a two-piece chassis comprising a rear enclosure 105 and an access door 110, which may be connected by one or more hinges 360. The device may also include a base 333. The base 333 may be made of one or more of a heat insulating material, a vibration-absorbing material, and/or a sound deadening material. Such a base 333 would facilitate placement of device 100 in a home or professional setting if desired.

A process 500 for presenting scents to a user is illustrated in FIG. 5, according to an embodiment. At 510, a scent is identified for presentation. This scent may be determined by the logic programmed in the control module. At 520, the belt is rotated until the scent reservoir associated with the determined scent is exposed in the isolation chamber. In some embodiments, belt 140 is rotated until the selected scent reservoir is in proximity to the opening of the lower drum 130. At 530, the scent reservoir is heated by the mechanism described above. This releases the determined scent into the lower drum (i.e., the isolation chamber). Activation of the fan pushes the scent out of the apparatus.

In some embodiments, belt 140 is stationary and an isolation chamber (i.e., lower drum 130) can be moved to a desired scent reservoir of belt 140 to selectively isolate the scent reservoir for the scent release/presentation process. This can include moving a heat source (i.e., heater block 190) to the desired scent reservoir of belt 140. Alternatively, the heat source can be stationary and can be coupled to the moveable isolation chamber via a flexible duct.

The release of the scent continues until the heating interval is completed. The length of the heating interval may be determined by the programming of the control module, and may be unique to the determined scent or may be the same for all scents. A determination may be made at 540 as to whether the heating interval has completed. If not, heating continues at 530.

If the heating interval has concluded, then at 550 the heating ends and the heating element is removed from the scent reservoir. This allows cooling of the scent reservoir. The length of the cooling interval may be determined by the programming of the control module, and may be unique to the determined scent or may be the same for all scents. A determination may be made at 560 as to whether the cooling interval has completed. If not, cooling continues at 550.

The cooling interval can be based on one or more of the size of the room in which the device is operating, characteristics of the scent (e.g., base, middle, or top notes as would be mapped on the fragrance pyramid), temperature and/or humidity in the room; and altitude (which may be determined by user's location, using, for example, global positioning coordinates or the user's internet protocol address), the device's fan speed and/or fan capacity (e.g., cubic feet per minute), user's input, and the distance between the device and the user in operation.

When the cooling interval has completed, a determination is made at 570 as to whether the entire schedule of scents has been completed. If not, the process returns to 510, where another scent is determined by the programming for presentation. The process 500 may repeat, so that a sequence of scents is presented to the user. Once this schedule has completed, as determined by the programming at 570, the process may stop at 580.

As noted above, the sequence of scents as presented to the user may or may not coincide with the arrangement of scent reservoirs on the belt. After a determined scent has been presented to the user, the next scent on the schedule may not correspond to the adjacent scent reservoir on the belt. In either case, the belt will be rotated until the reservoir having the next scent is positioned at the opening of the lower drum. The rotation of the belt to the correct position may be facilitated by the operation of the belt position sensor and the position indicators on the belt.

It can be desirable to isolate a given scent for presentation to a user, in that unintentional mixing of scents may diminish the therapeutic value of the apparatus and its operation. Preferably, a scent may be presented to a user, perhaps followed by an interval in which the scent dissipates prior to a next scent being presented. In an embodiment, this isolation may be facilitated by introducing an intervening odor that serves as a separator between successive scents. Such an intervening odor may serve to neutralize a preceding scent. Compounds that may be used for this purpose include hydroxypropyl cyclodextrin, diethylene glycol, sodium citrate, sodium maleate, or a combination of some or all of these. Alternatively, the intervening odor may be a mixture of odors that in combination create an olfactory white odor.

To implement such an intervening odor, one or more scent reservoirs 150 may hold scent source material that comprises such a mixture of odors, or one or more of the above compounds. Such scent source material may or may not be embodied in wax. The intervening odor may then be included in the schedule programmed into the logic of the control module. The apparatus would then present a scent from a particular scent reservoir, followed by presentation of an intervening odor from its scent reservoir, followed by presentation of another scent, etc. In various embodiments, presentation of an intervening odor may or may not require heating.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phrascology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A belt, comprising:
a band having a plurality of scent reservoirs, wherein each of the plurality of scent reservoirs releases a scent when heated.

2. The belt of claim 1, wherein each of the plurality of scent reservoirs contains a different scent.

3. The belt of claim 1, wherein adjacent scent reservoirs of the plurality of scent reservoirs have different scents.

4. The belt of claim 1, wherein each of the plurality of scent reservoirs comprises a scent source material that emits a vapor when heated.

5. The belt of claim 4, wherein the scent source material, as held in each scent reservoir, is in a mixture of wax.

6. The belt of claim 4, wherein the scent source material is held in each scent reservoir at least in part by a semi-permeable material that permits the vapor from the scent source material to escape.

7. The belt of claim 4, wherein the vapor from the scent source material escapes the scent reservoir upon heating of the scent source material.

8. The belt of claim 1, wherein the band comprises a continuous band.

9. The belt of claim 1, wherein the band comprises a plurality of belt sections, each belt section comprising a first attachment feature at a first end of the belt section and a second attachment feature at a second end of the belt section, wherein the first attachment feature of a first belt section is configured to attach to the second attachment feature of a second belt section.

10. The belt of claim 1, wherein the plurality of scent reservoirs are embedded in the band.

11. The belt of claim 1, further comprising one or more identifying markers configured to provide information on one or more scent reservoirs or a position of the belt.
